(19) United States
(12) Patent Application Publication
(10) Pub. No.: US 2022/0008282 A1
(43) Pub. Date: Jan. 13, 2022

(54) SHOCK WAVE APPARATUS AND METHOD FOR TREATING A HUMAN OR ANIMAL BODY

(71) Applicant: FERTON HOLDING S.A., Delémont (CH)

(72) Inventors: Gary EVANS, Allinges (FR); Nicolas SUMI, Lausanne (CH)

(21) Appl. No.: 17/293,110

(22) PCT Filed: Dec. 16, 2019
(86) PCT No.: PCT/EP2019/085333

§ 371 Date: May 12, 2021

(30) Foreign Application Priority Data
Dec. 21, 2018 (DE) 10 2018 133 356.0

(51) Int. Cl. A61H 9/00

(52) U.S. Cl. CPC A61H 9/0071; A61H 2201/1238; A61H 2201/0153

(57) ABSTRACT

A pressure wave device for the treatment of a human or animal body having a pneumatic drive for generating a pressure wave for coupling into the human or animal body, having at least one compressor for generating source gas and having a handpiece into which the source gas can be introduced and by means of which a pressure wave is generated, and having a pressure regulating device for adjusting a pressure wave generating pressure for generating the pressure wave, where the at least one compressor for generating source gas is adjustable in steps to at least two power levels, and the pressure regulating device regulates the pressure wave generating pressure (P_{D_i}) by adjusting the source gas pressure (P_{s_i}) at each power level, such that each power level is thereby preferably determined by a range of pressure wave generating pressure values and frequencies of the activation of the pressure waves, and the selection of a power level is preferably carried out using a table in which the respective ranges of pressure wave generating pressure values and frequencies are stored.

### TECHNICAL FIELD

The present disclosure relates to a pressure wave device for the treatment of a human or animal body with a pneumatic drive for generating a pressure wave for coupling into the human or animal body, and a method for treating a human or animal body with pressure waves with a pressure wave device described herein.

### BACKGROUND

Pressure wave devices for the treatment of the human or animal body with pressure waves, in which the impact of an accelerated impact part on an impact body generates a pressure wave, are known in themselves.

For example, pressure wave devices for the treatment of the human or animal body are known from EP 2 529 792 or EP 2 381 864 B1. The pressure wave devices have a pneumatic drive to generate a pressure wave. In this case, the pressure wave is generated by a percussion element that is pneumatically accelerated and strikes against a transmission element that couples the pressure wave into the body by contact. The pressure wave devices therefore have a compressor as part of the pneumatic drive for generating source gas, the compressor having a compressor motor. The pressure wave devices have a device for adjusting the pressure wave generating pressure, e.g. by means of the speed of the compressor motor, i.e. a device for adjusting the accelerating gas pressure. In the pressure wave device of EP 2 381 864 B1, the compressor operates continuously at different powers in order to be able to provide pressure waves of different strengths in a tunable manner in a handpiece, which leads to premature wear of the compressor and is associated with different loud background noises.

It is known that in a pressure wave device the compressor is controlled according to the gas pressure to be generated for the pressure wave. In this case, the compressor is usually operated until a predetermined maximum source gas pressure value, which in some variants is stored in a pressure reservoir, is reached. After the source gas pressure value has been reached, the compressor is usually switched off in order to counteract the generation of excess pressure. As a result, the compressor starts up again and again during treatment, which creates a background noise that is sometimes louder and sometimes quieter. This in turn has the consequence that a patient actively perceives this and feels disturbed in his well-being during the treatment by the background noise. The compressor also wears out relatively quickly.

In the case of pressure wave devices, it is desirable that they provide a constant pressure over a certain period of time in a handpiece, in particular in ranges of different flow rates, which can be adjusted by a user as required. In particular, it is desirable that a constant pressure is provided by the pressure wave device or pneumatic drive for a flow rate range determined by a switching frequency of a corresponding valve, such as a solenoid valve of the handpiece. This is a power range or load range. This is equally true for varying pressures at a fixed switching frequency or a combination of both settings. Furthermore, it is desirable that an acoustic background noise of the pressure wave device is minimized or constant during operation of the same, whereby the well-being of the human or animal patient can be increased, since a perception of the background noise in case of monotony of the same fades into the background for the patient. Finally, the service life of the compressor should be extended.

### BRIEF SUMMARY

The disclosure provides a pressure wave device in which the background noise during operation of the pressure wave device is reduced or constantly less perceptible, irrespective of the load at which the pressure wave device is currently calling up. The load is essentially dependent on the frequency of the pressure wave device, i.e. with which switching frequency a valve of the handpiece is operated (amount of air) as well as the pressure at which the pressure wave device is operated (available speed for the impact part to be accelerated). Furthermore, the disclosure provides a pressure wave device which delivers a regulated or adjustable, in particular variably constant pressure.

According to the disclosure, the pressure wave device for the treatment of a human or animal body comprises a pneumatic drive for generating a pressure wave for coupling into the human or animal body. Furthermore, the pressure wave device comprises at least one compressor for generating source gas (or “source gas”) with at least two adjustable power levels as well as a handpiece into which the source gas can be introduced via a connection line and by means of which an impact part can be accelerated in order to couple the pressure wave into the human or animal body. In addition, the pressure wave device comprises a pressure regulating device for adjusting the source gas pressure to a pressure wave generating pressure for generating the pressure wave. The pressure wave generating pressure is the pressure that acts directly on the impact part.

The pressure wave device has a handpiece into which the source gas can be introduced and by means of which a pressure wave is generated, and a pressure regulating device for adjusting a pressure wave generating pressure for generating the pressure wave, wherein at least one compressor, in particular of a compressor system for generating source gas, is adjustable in steps to at least two power levels, each power level is determined by a range of pressure wave generating pressure values (P_{D_Bi}) and frequencies (f_{Bi}), and the pressure regulating device regulates the pressure wave generating pressure (P_{D_i}) by adapting the source gas pressure (P_{s_i}) at each power level Lᵢ.

According to the disclosure, the pressure wave device has at least one compressor system which can be operated in stages in different operating states to generate different power levels. The selection of the different power levels can either be done by a pre-setting depending on the adjusted operating parameters of the pressure wave device (pressure wave generating pressure and frequency range) or the power level is changed independently by the pressure wave device when the pressure regulating device for adjusting the source gas pressure to a pressure wave generating pressure and/or for adjusting the desired flow rate, i.e. the frequency of the valve opening for repeated acceleration of the impact part, is no longer able to provide the pressure wave device with the required pressure wave generating pressure and/or the required flow rate.

In other words, the pressure wave device according to the disclosure comprises a compressor system operable in stages in different operating states for generating different power levels, wherein the pressure regulating device adjusts the pressure wave generating pressure by adapting a first source gas pressure within a first power level, i.e. within a first determined flow rate range, and wherein the pressure regulating device switches the compressor system into a second operating state with a second power level in order to adjust, in particular reduce, the pressure wave generating pressure as soon as the pressure regulating device is no longer able to provide the required pressure wave generating pressure and/or the required flow rate.

The pressure wave generating pressure is the pressure with which the impact part of the pressure wave device is accelerated. The flow rate is the frequency of the valve opening at the handpiece of the pressure wave device, i.e. the number of pressure waves generated per unit of time. A certain power range is composed of the desired pressure wave generating pressure at a certain flow rate. The higher the pressure wave generating pressure within a certain power range is supposed to be, the lower is the flow rate and vice versa. Within a certain power range, there is also a first maximum pressure wave generating pressure at a minimum flow rate and a maximum flow rate at a minimum pressure wave generating pressure. If, therefore, a pressure wave generating pressure is to be generated that exceeds this maximum pressure wave generating pressure in this particular power level or if a flow rate is to be achieved that exceeds the maximum flow rate in this particular power level, the pressure wave device automatically changes the power level.

Each power level (Lᵢ) is determined by a range of pressure wave generating pressure values (P_{D_Bi}) and frequencies (f_{Bi}) of the activation of the pressure waves. Advantageously, the selection of a power level (Lᵢ) is made on the basis of a table in which the respective ranges of pressure wave generating pressure values (P_{D_Bi}) and frequencies (f_{Bi}) of the activation of the pressure waves are stored.

The pressure regulating device is designed to adjust different pressure wave generating pressures P_{D} for generating the pressure wave. For this purpose, for example, the pressure regulating device for adapting the source gas pressure, in particular for providing a substantially constant, predetermined pressure wave generating pressure P_{D}, is arranged in the handpiece immediately in front of or at an inlet of the handpiece. In other words: While the compressor and/or the compressors in a discretely controllable operating state cover a certain performance level at constant speed, in which a current source gas pressure is subject to, for example, operational or load-dependent fluctuations, it is envisaged that an adjustment of the pressure wave generating pressure by means of the pressure regulating device takes place at constant speed. Preferably, the compressor system is designed such that the discretely adjustable power levels do not overlap. For example, the compressor system may consist of a single compressor or several compressors.

It is also conceivable to arrange the pressure regulating device in the pneumatic drive or at any position in the connection line. In this context, the term “substantially constant pressure wave generating pressure” means a gas pressure which deviates by at most ±0 to 0.5 bar from the predetermined pressure wave generating pressure P_{D}, i.e. the predetermined set value of the gas pressure for the pressure wave to be generated. Preferably, the pressure regulating device is designed so that the deviations from the desired pressure wave generating pressure, i.e. a setting accuracy of the pressure wave generating pressure, are always smaller than 0.2 bar, preferably smaller than 0.15 bar and particularly preferably smaller than 0.1 bar. This makes it possible to achieve the adjustment accuracy required for reliable use of the pressure wave device.

The same applies mutatis mutandis to the corresponding flow rates that must be available at certain pressure wave generating pressure values.

Preferably, the compressor system comprises multiple compressors that are switched between, for example, to set a desired capacity level. In other words, the individual compressor is not operated in a particular operating state to control or set the source gas pressure range, but rather is switched between (or these are individually switched in) the compressors, each of which is responsible for producing a single operating state with a corresponding performance level. According to the disclosure, the respective power level is defined by the operating parameters pressure and frequency (flow rate) of the pressure wave device, which determine the consumption (amount of air per time unit) of pneumatic means of the pressure wave device.

The pressure regulating device can be used to adjust a predetermined pressure wave generating pressure P_{D}, for example before the gas pressure is fed into the handpiece to accelerate an impact part there and generate a pressure wave. This has the advantage that a pressure wave generating pressure range can be provided for a certain power level, e.g. a certain flow rate range, independent of a concretely used switching frequency of a valve, such as a solenoid valve, in the handpiece, i.e. independent of the activation duration of the pressure wave generating pressure and/or independent of a compressor power of a compressor or a currently induced source gas pressure by the compressor(s).

Furthermore, one or more corresponding compressors for generating the source gas pressure can be operated continuously in a certain power range, preferably during a treatment, so that a monotonous background noise is audible. Furthermore, the operating state of the compressors can be selected in such a way that a particularly advantageous background noise is produced. In particular, only those speeds can be selected at which strong vibrations or resonances do not occur. In the case of discretely adjustable speeds, the vibration of the pressure generating device can be specifically suppressed by constructive measures. Therein, the speed of the compressor or compressors is essentially determined by its voltage or operating voltage, which is discretely adjustable. Likewise, it is conceivable that an electrical control fixes the speed of the compressor to one of several fixed preselectable values. The discrete power levels of the compressor(s) are thereby assigned to fixed speeds of the compressor(s). This can in particular reduce a subjective perceptibility of the background noise during the operation of the pressure wave device, which in turn can increase the well-being of a patient during treatment. Due to the constant speed, the patient is less stressed than when the compressor is repeatedly switched on/off or when the speed is repeatedly changed. In other words, the compressor(s) operate at a fixed voltage at a fixed speed, the load differences (pressure and flow rate) merely cause the motors to draw different amounts of current.

Preferably, the pressure regulating device comprises a drain valve comprising in particular a valve, in particular a solenoid valve. For example, the pressure regulating device comprises a proportioning valve. A proportioning valve allows, for example, continuous transitions between a fully open and a fully closed state. This can be, for example, an electromagnetically controlled or a medium-controlled proportioning valve.

The pressure regulating device is advantageously used to provide a constant pressure on or in the handpiece, namely the pressure wave generating pressure P_{D} for generating the pressure wave, so that uniform pressure waves can be introduced via the handpiece into the body to be treated.

In general, a pressure reducer is understood to be a pressure valve for installation in a hose or pipe system, wherein the pressure valve, despite different pressures on an input side (input pressure), in this case corresponding to the source gas pressure Pₛ, ensures that a certain output pressure, in this case corresponding to the pressure wave generating pressure P_{D}, is not exceeded on the output side, which in this case leads into the handpiece. By providing a pressure regulating device, the source gas pressure Pₛ is converted to the required pressure wave generating pressure P_{D} for generating the pressure wave. A pressure reducer thus has the advantage that uniform pressure waves can be generated in the pressure wave device.

According to an embodiment of the pressure wave device, the pressure regulating device has at least one pressure sensor for determining a pressure wave generating pressure P_{D}. This makes it possible to check in a simple manner whether the pressure regulating device has carried out an appropriate regulation, in particular a pressure reduction. Preferably, the pressure regulating device is designed as a control loop. It is conceivable to provide a further pressure sensor. By arranging a further pressure sensor, the gas pressure, i.e. the source gas pressure, can be detected for checking, in particular before entry into the handpiece. Depending on the detected source gas pressure and/or depending on the detected pressure wave generating pressure P_{D}—depending on where the at least one pressure sensor is arranged—a pressure wave generating pressure P_{D}, in particular stored in a control system, can be set.

In particular, it is envisaged that the compressor system is controlled or configured such that the discrete power level always provides a source gas pressure that can be reduced to the desired pressure wave generating pressure to ensure that it can be provided at the handpiece reliably and independently of the switching frequency of the pressure wave generating pressure. In other words, it is avoided that the provided source gas pressure is not sufficient to realize the desired pressure wave generating pressure at all times, i.e. within a flow rate range.

According to one embodiment of the pressure wave device, a predetermined range of pressure wave generating pressure values P_{D_i} and a predetermined range of switching frequencies fᵢ with i≥1 is stored in a controller for a power level Lᵢ with i≥1, i.e. a pressure table, wherein the pressure regulating device is designed to adjust the necessary power level Lᵢ of the compressor system as a function of the desired pressure wave generating pressure P_{D_i}, e.g. for a desired range of switching frequencies fᵢ.

As proposed herein, a first source gas pressure P_{s_i} with i≥1 within a certain power level is first generated by means of one or more compressors, which in particular can then be detected by a pressure sensor. The detected source gas pressure P_{s_i} can be matched or compared with predefined or desired pressure wave generating pressures P_{D_i}, which are stored in a control system. A range of pressure wave generating pressures P_{D_i} is assigned to each source gas pressure value P_{s_i}, in particular at a predetermined frequency of the pressure wave device, i.e. time-dependent number of strokes of the impact part, for generating the pressure waves. Such an allocation takes place in particular in dependence of an adjusted frequency at the pressure wave device. The pressure wave generating pressure P_{D_i} is thus variable, wherein variable is to be understood in the present case as meaning that, on the basis of a certain predefined, in particular adjusted, source gas pressure range or load range, which in each case has a maximum source gas pressure value P_{s_i}, a desired pressure wave generating pressure P_{D_i} can be adjusted, which is assigned to a specific range of predetermined, desired pressure wave generating pressures P_{D_i}, and another certain source gas pressure range or load range, which in each case has a different maximum source gas pressure value P_{s_i±1}, is assigned to another range of pressure wave generating pressures P_{D_i±1}.

Depending on the specific, stepwise predetermined load ranges or power levels Lᵢ, the desired pressure wave generating pressure P_{D_i} or another desired pressure wave generating pressure P_{D_i±1} is set at another power level L_{i±1} by means of the pressure regulating device, at specific flow rates. This has the advantage that uniform pressure waves with a desired pressure wave generating pressure P_{D_i} can be generated during a treatment, by means of stepwise controlled, discrete power levels Lᵢ. The compressor(s) thus operates in discrete stages.

Preferably, the pressure wave device has at least two compressors. Each of the at least two compressors can be operated at the same or a different speed level, in particular at different power levels. This allows the compressors to be operated at different, in particular discrete, power levels. In the present case, a discrete power level can be realized at a predetermined source gas pressure and a predetermined flow rate. The at least two compressors in particular achieve that a noise, i.e. a background noise, of the pressure wave device is reduced, in particular when the pressure wave device is used in a range of a low flow rate and/or at a low source gas pressure. This is because, for example, two or more compressors can be used to generate a certain source gas pressure P_{s_i}, each of which is operated at a lower power.

Preferably, the at least two compressors are connected to one or are each connected to one compressor motor for controlling the compressors. The compressor or each compressor can thus be controlled by the corresponding motor at a predetermined power or a predetermined frequency, whereby a predetermined power level can be set at the corresponding compressor. Advantageously, this can reduce a constantly changing noise of the pressure wave device and the wear is reduced by a constant operation.

According to a preferred embodiment of the pressure wave device, a cold trap for collecting cooled, condensed source gas is arranged, in particular between the at least one compressor and the pressure regulating device. Consequently, condensed gas, in particular condensed air, can be collected by the cold trap. This prevents condensed gas, i.e. a liquid, from being fed into the handpiece. In this way, only source gas is fed into the handpiece. In the long term, this prevents moisture from settling in the handpiece, which could lead to mold, corrosion or the like.

Further preferably, in particular between the compressor or compressors and the pressure regulating device, a relief valve is provided for discharging an overpressure of the source gas. This can prevent the pressure wave device from being damaged or even destroyed by a generated overpressure. The overpressure valve is used especially at a gas pressure greater than 5 bar, preferably at a pressure of up to 25 bar.

Preferably, each compressor is designed to generate different, in particular discrete, volume flows in stages in order to generate different source gas pressures and/or flow rates of the source gas. Consequently, each compressor can be adjusted or regulated with regard to its compressor power, so that each compressor can generate different volume flows in stages and thus generate different source gas pressures and/or flow rates of the source gas. The compressor power is thus used to generate a source gas at a stepped, i.e. discrete, speed of the compressor motor, Preferably, each compressor has its own compressor motor. In particular, each compressor has a direct current (DC) motor. This allows each compressor to be operated at a fixed speed (i.e. fixed predetermined voltage) but with different current consumption at different pressure values and/or different flow rates within one power level. This has the advantage that a background noise of the pressure wave device can be reduced overall.

According to a preferred embodiment of the pressure wave device, the pressure wave device is configured in such a way that the pressure wave device can be vented by means of a controllable venting valve, in particular a solenoid valve, after a, in particular every, use of the pressure wave device. Via the venting valve, a source gas remaining in a circuit of the pressure wave device can be removed from the circuit in an advantageous manner. The venting valve preferably operates autonomously. However, it is conceivable that the vent valve can be additionally controlled by a controller. In particular, such a venting valve can prevent source gas from settling in the circuit or the pressure wave device in the long term and possibly condensing. In this way, the growth of bacteria or the like can be counteracted in a simple manner.

Another aspect of the present disclosure relates to a method of treating a human or animal body with pressure waves, in particular generated with a pressure wave device described herein, the method comprising the steps of:
starting the at least one compressor to generate source gas,
passing the source gas from the compressor to the handpiece, wherein the at least one compressor is set to a discretely controllable operating state to which a discrete power level L is assigned,
wherein the pressure wave generating pressure is regulated by adapting a first source gas pressure within the power level L by a pressure regulating device at a certain flow rate,
and wherein the pressure wave generating pressure is regulated by increasing or by decreasing the power level L.

When the compressor or compressors are switched on, a first pressure of the source gas P_{s_i} with i≥1 within a discrete power level L can be adjusted. The possible predetermined or desired pressure wave generating pressures P_{D_i} with i≥1, which are each assigned to a specific discrete power level Lᵢ at a specific flow rate or frequency fᵢ, can in particular be stored in a control system and displayed to a user for individual selection when the pressure wave device is switched on. It is also possible for a user to manually enter a predetermined pressure wave generating pressure P_{D_i}. In this case, a first source gas pressure P_{s_i} can then initially be generated at a first power level Lᵢ, which can be reduced to a first pressure wave generating pressure P_{D_i} by means of the pressure regulating device. The adjusted pressure wave generating pressure P_{D_i} can then flow to an inlet of the handpiece. While the source gas flows to the handpiece, the gas pressure is detected, in particular by a pressure sensor. Depending on where the pressure sensor is arranged, it detects either the source gas pressure (before passage of the pressure regulating device) or the pressure wave generating pressure (after passage of the pressure regulating device) or both pressures are measured with two sensors. Consequently, depending on the arrangement of the at least one pressure sensor, the source gas pressure P_{s_i} or the pressure wave generating pressure P_{D_i} is detected. The pressure regulating device is controlled depending on the detected source gas pressure P_{s_i} of the source gas or the pressure wave generating pressure P_{D_i}. This means that, on the one hand, the handpiece is always supplied with a constant pressure wave generating pressure P_{D_i} so that uniform pressure waves can be generated at one end of the handpiece for transmission into the human or animal body, on the other hand the compressor(s) do not always operate at full load but only constantly in a range, i.e. at the discrete power level Lᵢ, in which the required pressure wave generating pressure P_{D_i} falls.

Preferably, the method further comprises the steps of:
closing a vent valve, and/or
at least partially opening or at least partially closing a pressure regulating device or regulating the pressure regulating device for passing the source gas from a compressor to a handpiece.

In one step, the venting valve is closed to prevent a source gas generated, in particular in the at least one compressor, which is intended to generate the pressure wave, i.e. the source gas pressure P_{s_i} with i≥1, from escaping from the pressure wave device. Furthermore, the pressure regulating device can be at least partially opened or partially closed so that a generated source gas, after a pressure reduction or a pressure increase, and thus a pressure wave generating pressure P_{D_i}, can flow to the handpiece.

Preferably, after a detection of the actually generated pressure wave generating pressure, it is checked whether the detected pressure wave generating pressure corresponds to a desired or predetermined pressure wave generating pressure and, depending on a difference between detected pressure wave generating pressure and desired pressure wave generating pressure
a) the state of the pressure regulating device is maintained unchanged, provided that the difference is approximately zero;
b) the pressure regulating device is at least partially opened or the source gas pressure P_{s_i} is reduced, provided that the difference is positive, i.e. the detected pressure wave generating pressure is above the desired pressure wave generating pressure, and
c) the pressure regulating device is at least partially closed or the source gas pressure P_{s_i} is increased, provided that the difference is negative, i.e. the detected pressure wave generating pressure is below the desired pressure wave generating pressure.

Here, the source gas pressure P_{s_i} is increased or decreased or maintained, depending on an evaluation of the difference between the detected pressure wave generating pressure and the desired pressure wave generating pressure. The detected pressure wave generating pressure P_{D_i} results from the source gas pressure P_{s_i} generated at the at least one compressor. Each pressure wave generating pressure P_{D_i} is assigned to a power level Lᵢ, i.e. a constant speed range of the compressor(s).

When a pressure wave generating pressure P_{D_i} is detected to be above a desired pressure wave generating pressure P_{D_i}, i.e. a predetermined pressure wave generating pressure PD_i, the pressure regulating device is at least partially opened or further opened to discharge source gas, thereby reducing the pressure wave generating pressure P_{D_i}. If a pressure wave generating pressure P_{D_i} below the desired pressure wave generating pressure P_{D_i} is detected, the pressure regulating device is at least partially closed or further closed to generate a higher pressure wave generating pressure P_{D_i}. If, on the other hand, a pressure wave generating pressure P_{D_i} substantially equal to the desired pressure wave generating pressure P_{D_i} is detected, the state of the pressure regulating device is maintained unchanged.

Consequently, by opening or closing the pressure regulating device, in particular the proportioning valve, alone, the desired pressure wave generating pressure P_{D_i}, can be adjusted, whereby opening or closing the pressure regulating device does not require that the source gas pressure P_{s_i} generated by the at least one compressor has to be changed. However, the source gas pressure P_{s_i} can be changed if necessary, in particular by adjusting the at least one compressor to a different power level. The “necessity” arises when the difference is so great that the desired pressure wave generating pressure P_{D_i} can only be achieved if another (higher or lower) stage of the source gas pressure P_{s_i} is set. The same applies mutatis mutandis to changes in the flow rates.

In the present case, the term “substantially equal” or the term “difference approximately zero” means a pressure wave generating pressure P_{D_i} which deviates by at most ±0 to 0.5 bar from the specified pressure wave generating pressure P_{D_i}, i.e. the specified setpoint value of the pressure wave generating pressure P_{D_i}. For example, a range of adjustable pressure wave generating pressures P_{D_i}, in particular as a function of a frequency, can be stored in the control system for a source gas pressure P_{s_i}. In this way, a step model for the source gas pressure P_{s_i} to be generated is realized in a simple manner.

An “opening” or “closing” of the pressure regulating device is only explained by way of example. It is conceivable that the pressure regulating device works in the opposite direction, so that opening or closing the pressure regulating device has the opposite effect as described here. “Opening”/“closing” also includes other controls for adjusting a pressure.

Preferably, the at least one power level is incrementally increased or decreased when P_{s_(i-1)}≤P_{D_i} applies to the maximum source gas pressure at a certain power level Lᵢ₋₁ or P_{D_i}≤P_{s_(i-1)} applies to the required or necessary pressure wave generating pressure, wherein adapting the compressor speed(s) and/or the addition of further compressors or the switching off of individual compressors takes place for an increase or decrease of the power level. It is conceivable to store an allocation of a range of possible pressure wave generating pressures P_{D} at a certain flow rate or frequency f or for a certain frequency range in a control system in order to define the power level L. Furthermore, a voltage U to be set can be stored in the control with which the at least one compressor must be operated in order to achieve the desired source gas pressure Pₛ at a set power level in order to achieve the desired pressure wave generating pressure P_{D} at the desired frequency. In particular, a voltage at the compressor can be selected for a pair of pressure wave generating pressure and frequency selected by the user. For example, it is conceivable that a control device automatically adjusts the voltage at the compressor based on the desired pressure wave generating pressure and the set frequency, which are entered by the user at a man-machine interface, which prompts a power range which in turn comprises the desired pressure wave generating pressure. In this way, it is advantageously possible to select an operating state for the compressor that is particularly beneficial for low wear and low background noise, since the compressor or compressors are always operated at a constant speed within a power level L at a fixed voltage. In particular, it is also conceivable that the compressor is appropriately optimized or designed for the discretely controllable voltages in order to ensure comparatively quiet operation and a long service life.

Further preferably, the setting of the predetermined pressure wave generating pressure can be changed in the proposed method, in particular during a treatment. When, for example, the predetermined pressure wave generating pressure is changed during a treatment, the pressure regulating device is further opened or further closed when a pressure wave generating pressure adapted to the changed pressure is exceeded or undershot. The pressure regulating device is thus activated as a function of the detected pressure of the source gas when the detected pressure wave generating pressure no longer corresponds to the predefined or the changed predefined pressure wave generating pressure, in particular no longer corresponds within predefined error limits of the pressure wave generating pressure.

Further preferably, opening and/or closing of the pressure regulating device also takes place depending on a frequency at which the pressure wave device is operated. Depending on the flow rate, i.e. how often within a unit of time the impact part of the pressure wave device is accelerated at a predetermined pressure wave generating pressure, more or less air is consumed, which is regulated by the pressure regulating device. Only when the pressure regulating device no longer achieves a required flow rate or frequency at a given pressure wave generating pressure, or this can also be achieved at a lower power level, is the compressor(s) controlled to a higher or lower power level.

Further preferably, a certain power level Lᵢ is stored in a control system for a range of pressure wave generating pressures P_{D_i} and a range of frequencies fᵢ, to which a fixed voltage is assigned, which triggers a certain speed at the compressor or at the compressors or successively switches compressors on or off in order to carry out a process as described herein. The control system may further be adapted to control the various valves, in particular, inter alia, the venting valve. Preferably, discrete values of the source gas pressure P_{s_i} are also stored in the control system, which, for a predetermined pressure wave generating pressure P_{D_i}, specify a voltage and/or a frequency for activating the compressors, and/or a frequency at which, for example, the valves in the handpiece switch or the at least one compressor is to be operated.

Further preferably, for carrying out the method, the at least one compressor of the pressure wave device has a plurality of adjustable, stepwise power levels with which the at least one compressor can be operated in order to generate a source gas pressure P_{s_i}. The respective power level can be set in particular by determining a voltage which is associated with the desired pressure wave generating pressure and the set frequency. It is conceivable that the at least one compressor generates different source gas pressures at different power levels, such as for example a source gas pressure P_{s_i} of 2 bar, 5 bar, 8 bar or 10 bar at six different power levels. By means of the pressure regulating device, a pressure wave generating pressure P_{D_i} of 0-2 bar, 2-5 bar, 5-8 bar or 8-10 bar can then be generated at certain frequencies. An assignment of the different power levels, with the maximum possible source gas pressures P_{s_i}, to the corresponding desired pressure wave generating pressure P_{D_i} at a certain frequency can exemplarily be taken from the following table, which shows in particular a reduction of the source gas pressure to the desired pressure wave generating pressure:

| Power level | 1 | 2 | 3 | 4 | 5 | 6 |
|---|---|---|---|---|---|---|
| Maximum source gas | 2 | 5 | 5 | 8 | 8 | 10 |
| pressure Pₛ_{_{—}}ᵢ [bar] | | | | | | |
| Maximum pressure wave | 0-2 | 2-5 | 2-5 | 5-8 | 5-8 | 8-10 |
| generation pressure P_{D}_{_{—}}ᵢ | | | | | | |
| [bar] | | | | | | |
| Frequency f [Hz] | 1-10 | 1-10 | 10-60 | 1-10 | 10-60 | 1-60 |

### BRIEF DESCRIPTION OF THE DRAWINGS

Further advantages and features of the present disclosure will be apparent from the following description of preferred embodiments with reference to the accompanying figures. It is understood that individual embodiments shown in the respective figures may have features that can also be used in other embodiments, even if this is not explicitly stated, and unless this has been excluded due to technical circumstances or explicitly. It is shown in:

FIG. 1 a pressure wave device according to the present disclosure,

FIG. 2 a circuit diagram of the pressure wave device according to FIG. 1,

FIG. 3 an alternative circuit diagram of the pressure wave device according to FIG. 1,

FIG. 4 a flow chart of a proposed method for treating a human or animal body with pressure waves, and

FIG. 5 a visualization of the step model proposed herein, which shows a relationship between a pressure wave generating pressure P_{D_i} and a frequency at which the at least one compressor is operated.

### DETAILED DESCRIPTION

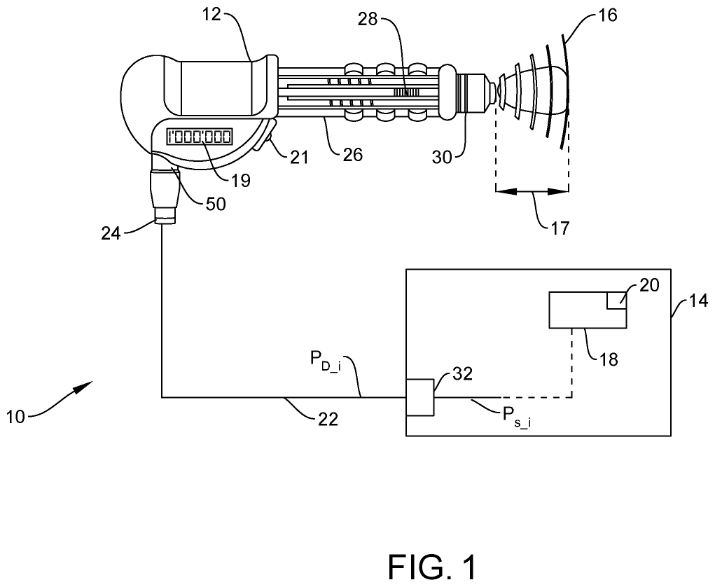

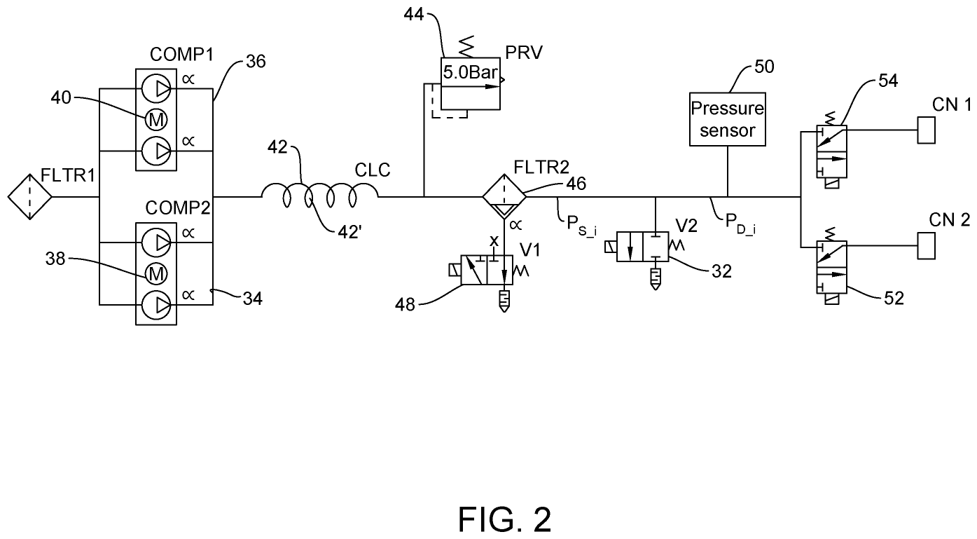

FIG. 1 shows a pressure wave device 10 for the treatment of a human or animal body (not shown) with a pneumatic drive 14 for generating a pressure wave 16 for coupling into the human or animal body. The pressure wave device 10, in particular the pneumatic drive 14, has at least one compressor 18 for generating source gas at a source gas pressure P_{s_i}, the compressor 18 comprising a compressor motor 20. It is conceivable that instead of one compressor 18, several compressors 34, 36 (FIG. 2) are comprised by the pressure wave device 10. Furthermore, the pressure wave device 10 has a handpiece 12, into which the source gas can be introduced via a connection line 22 into an inlet 24 of the handpiece 12. The pressure wave 16 can be coupled into the human or animal body via the handpiece 12. The handpiece 12 has an elongated guide tube 26 in and along which a projectile 28 can be accelerated by the source gas to an impact body 30. The impact body 30 serves as a transmission element for transmitting the pressure wave 16 from the handpiece 12 into the human or animal body. The pressure wave 16 is generated by the accelerated movement of the projectile 28 in the guide tube 26. The guide tube 26 thus serves as an acceleration path for the projectile 28. The pressure waves 16 generated by means of the pressure wave device 10 can penetrate up to 50 mm, into the human or animal body.

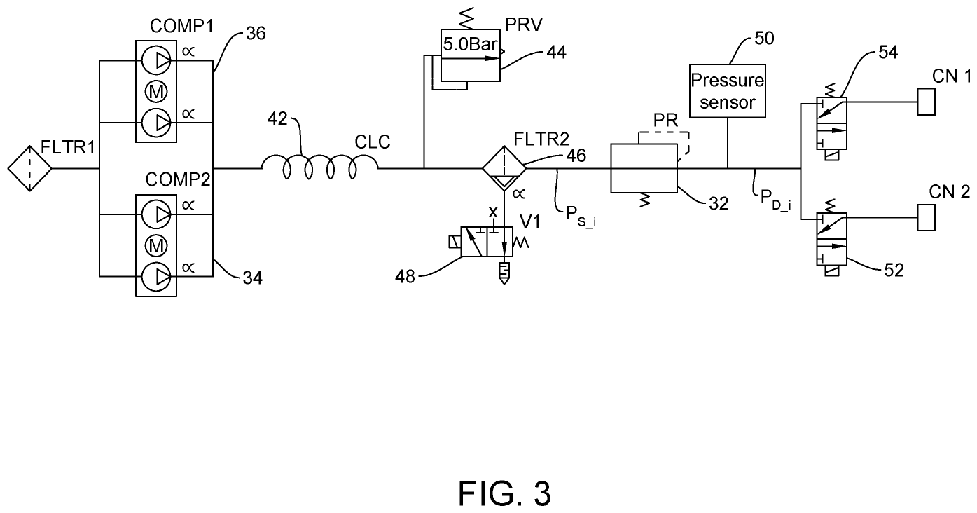

In FIG. 1, it can be seen in conjunction with FIGS. 2 and 3 that a pressure regulating device 32 is arranged in front of or at the inlet 24 of the handpiece 12 or optionally at the drive 14 for adjusting the source gas pressure P_{s_i}, in particular for providing a substantially constant pressure wave generating pressure P_{D_i}, in the handpiece 12. By means of a regulator 19, the pressure wave generating pressure predetermined at the input or the desired pressure wave generating pressure is adjustable and by means of an activation button 21, the frequency of the introduction of source gas accelerating the projectile is adjustable. By means of the compressor or a compressor system, a certain power level Lᵢ is controlled, whereby a first range of pressure wave generating pressures P_{D_i} can be adjusted by the regulator 19 and a first range of frequencies fᵢ can be set by the activation button 21 on the handpiece 12. Here, the source gas pressure P_{s_i} denotes a gas pressure generated by the compressor 18 or the plurality of compressors 34, 36 and prevailing before flowing through the pressure regulating device 32. A pressure wave generating pressure P_{D_i}, on the other hand, results from the source gas pressure P_{s_i} after flowing through the pressure regulating device 32.

FIGS. 2 and 3 each show an alternative of a circuit diagram of the pressure wave device 10 according to FIG. 1. According to FIG. 2, the pneumatic drive 14 of the pressure wave device 10 comprises two compressors 34, 36 instead of one compressor 18 with a compressor motor 20. Each compressor 34, 36 is connected to a compressor motor 38, 40 which drives the respective compressor 34, 36. The compressors 34, 36 thus serve to compress the source gas, wherein the source gas, after its compression, passes through a cooling zone in which a cooling device 42, in particular a cooling coil 42′, is arranged. In the cooling device 42, in particular in the cooling coil 42′, the compressed source gas is cooled before it is fed into the handpiece 12 via the connection line 22.

Furthermore, a pressure relief valve 44 is arranged between the cooling device 42 and the inlet 24 of the handpiece 12 for discharging an excess pressure of the source gas. The pressure relief valve 44 is designed to automatically discharge a pressure which exceeds a predetermined pressure, in particular a maximum source gas pressure P_{s_i}, from the connection line 22 or even before the source gas enters the connection line 22. This can prevent an overpressure from occurring in the handpiece 12. The pressure relief valve 44 is designed, for example, to automatically release a pressure greater than, for example, 5 or 10 bar, in particular from the connection line 22. Of course, it is conceivable to calibrate the pressure relief valve 44 to a different switching threshold of the gas pressure at which the pressure relief valve 44 releases gas pressure, if necessary. Preferably, the pressure relief valve is designed to withstand a gas pressure of up to 25 bar.

Particularly preferably, a cold trap 46 is arranged in the connection line 22 for collecting condensed source gas, i.e. the condensate. It is conceivable that the pressure relief valve 44 is arranged between the cooling device 42 and the cold trap 46. The cold trap 46 may further be connected to a drain or venting valve 48, such as a solenoid valve. The venting valve 48 may, in particular, allow condensate to be removed from the pressure wave device 10 and vented after each use of the pressure wave device 10. This ensures that no residual condensate and source gas remains in the pressure wave device 10 after a treatment. Furthermore, according to the disclosure, a pressure regulating device 32, in particular a pressure regulator, is arranged at or in front of the inlet 24 of the handpiece 12, which adjusts, i.e. regulates, the source gas pressure P_{s_i} in the connection line 22 to a predetermined, i.e. desired, pressure value, i.e. a pressure wave generating pressure P_{D_i}. It is further conceivable that the pressure regulating device 32 is arranged in the pneumatic drive 14 and the gas pressure is regulated before it flows into the connection line 22.

To check the pressure wave generating pressure P_{D_i} set via the pressure regulating device 32, a pressure sensor 50 is also arranged, for example in the connection line 22 or at another suitable position. The pressure sensor 50 is designed to detect the pressure wave generating pressure P_{D_i}, which flows in the connection line 22 after passing the pressure regulating device 32, and to indicate this to a user, e.g. on a display.

FIGS. 2 and 3 differ only in the design of the pressure regulating device 32. According to FIG. 2, the pressure regulating device 32 is designed as a valve, in particular as a solenoid valve, which can withstand a pressure of, for example, up to 7 bar, preferably up to 10 bar. According to FIG. 3, the pressure regulating device 32 is designed as a pressure reducer which can withstand a pressure of, for example, up to 7 bar, preferably up to 10 bar. A pressure reducer is essentially any pressure-reducing valve which is designed for installation in a hose or pipe system and which, despite different pressures on the input side (input pressure, in this case the source gas pressure P_{s_i}), ensures that a certain output pressure (in this case the pressure wave generating pressure P_{D_i}) is not exceeded on the output side. In particular, a drain valve differs from a pressure reducer in that its task is to reduce excess pressure. This is done, for example, by opening a valve and discharging gas or liquid, e.g. into the environment. The valve can be actively controlled or it can open automatically as an independent unit by means of a spring mechanism at a pressure to be set. In the pressure reducer, on the other hand, no medium is released. The spring mechanism in the pressure reducer always releases just enough opening cross-section to ensure that a pre-set pressure is achieved downstream of the pressure reducer.

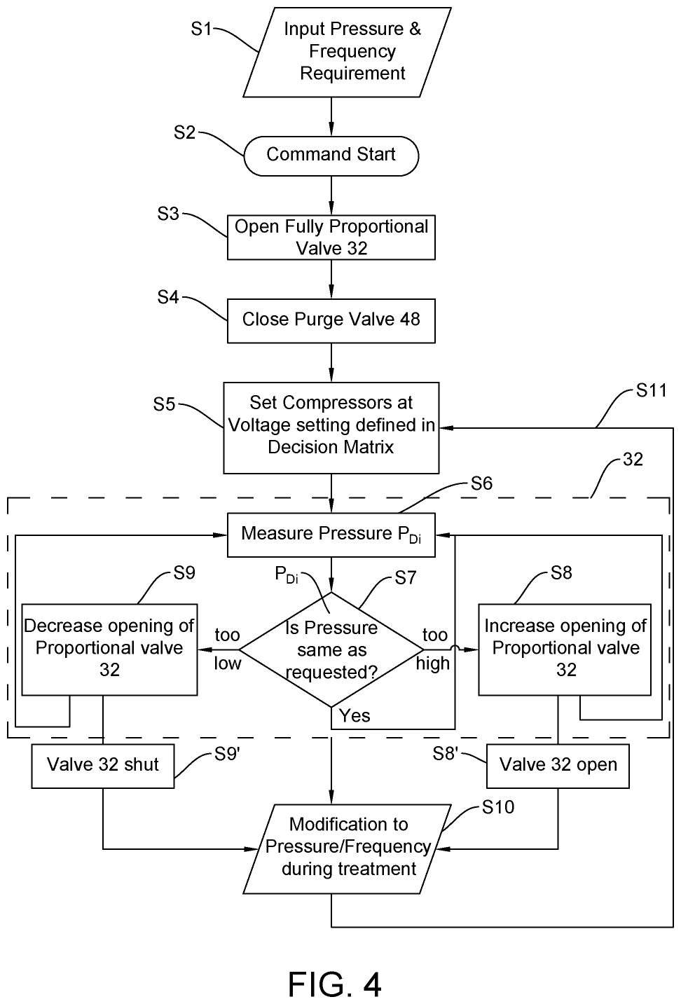

FIG. 4 shows a flow chart of a preferred method of treating a human or animal body with pressure waves 16 using a pressure wave device 10 as described herein. The method comprises the steps:
starting the at least one compressor 18, 34, 36 to generate source gas,
passing the source gas from the at least one compressor 18, 34, 36 to the handpiece 12, wherein
the at least one compressor 18, 34, 36 is operated in an operating state that produces a first source gas pressure within a source gas pressure range at a first power level,
a desired pressure wave generating pressure P_{D_i} is generated by adjusting the first source gas pressure by opening or closing a pressure regulating device 32 accordingly.

In the method proposed herein, as shown in FIG. 4 in a first step S1, inputs can first be made for the power level to be controlled, i.e. a maximum desired pressure wave generating pressure and a maximum desired frequency are entered or these are already stored for the corresponding power level in the pressure wave device 10. In particular, a specific voltage, especially one of several discretely selectable voltages, can be set for the compressor for a pressure wave generating pressure range to be controlled at a specific frequency (specified by the user). The selection allows, in particular, a low-wear and low-noise operating state to be selected for the compressor. After entering this information, the method as such can be started in a second step S2.

In a third step S3, the pressure regulating device 32 is first opened so that the generated source gas pressure P_{s_i} from the compressor or compressors 34, 36 can be conducted unhindered through the connection line 22. In a fourth step S4, the venting valve 48, which in an open state serves to vent the cold trap 46, is closed simultaneously or subsequently. In a fifth step S5, the at least one compressor 18, 34, 36 can then be controlled with the predetermined voltage U. The predetermined voltage can be stored in a control system. The optimum voltage U for low-wear and/or low-noise operation is preferably stored in relation to a predetermined discrete power stage Lᵢ. After switching on the at least one compressor 18, 34, 36, the generated source gas pressure consequently flows via the connection line 22 to the inlet 24 of the handpiece 12.

When flowing through the connection line, the source gas is regulated by the pressure regulating device 32 from a source gas pressure P_{s_i} to the desired pressure wave generating pressure P_{D_i}. For example, a source gas pressure P_{s_i} of 2 bar, which is assigned in particular to a first power level L₁ of the at least one compressor 18, 34, 36, can be assigned to a first range 1 of pressure wave generating pressures P_{D_i} and a first range of frequencies fᵢ. Here, it is now conceivable that the pressure regulating device 32 performs a reduction in order to achieve a pressure wave generating pressure P_{D_i} in a range between 1 bar≤P_{D_i}≤2 bar. It is further conceivable that, in addition to a first range 1, a second and a third range 2, 3 are provided to set a desired pressure wave generating pressure P_{D_i} at a desired frequency f to a second or third maximum source gas pressure P_{s_i}. The number of ranges for setting the pressure wave generating pressure P_{D_i} is not limited to three ranges 1, 2, 3. Rather, more or fewer ranges can be provided. The number of ranges depends in particular on the number of adjustable power levels of the at least one compressor.

By means of a pressure sensor 50, which is arranged, for example, in front of or at the inlet 24, the current pressure wave generating pressure P_{D_i} is detected after passing the pressure regulating device 32 in the communication line 22. If it is determined in a seventh step S7 by forming a difference between the detected pressure wave generating pressure P_{D_i} and the desired pressure wave generating pressure P_{D_i} that the detected pressure wave generating pressure P_{D_i} corresponds to a desired pressure wave generating pressure P_{D_i}, the settings on the pressure regulating device 32 remain unchanged and the pressure wave generating pressure P_{D_i} is further detected at a time interval according to the sixth step S6. If, on the other hand, it is determined in the seventh step S7 that the measured pressure wave generating pressure P_{D_i}, in particular in the connection line 22, exceeds the predetermined pressure wave generating pressure P_{D_i}, i.e. is too high, then in an eighth step S8 the pressure regulating device 32 is opened to such an extent that an increased pressure wave generating pressure P_{D_i}, in particular from the connection line 22, can escape (an opening of the pressure regulating device 32 is reproduced by step S8′). This can prevent an increased gas pressure, namely an increased pressure wave generating pressure P_{D_i}, from entering the handpiece 12 and being undesirable for the treatment.

Further, according to the sixth step S6, the pressure wave generating pressure P_{D_i} in the connection line 22 is detected by means of the pressure sensor 50. If it is determined in the seventh difference-forming step S7 that the desired pressure wave generating pressure is below the detected pressure wave generating pressure P_{D_i}, i.e. the predetermined pressure wave generating pressure P_{D_i} is too low, the pressure regulating device 32 is at least partially closed (a closing of the pressure regulating device 32 is reproduced by step S 9′). This ensures that less gas is blown off, so that an increased pressure wave generating pressure P_{D_i} occurs in the connection line 22, in particular at or in front of the inlet 24 of the handpiece 12.

Steps S7 to S9 ensure that the pressure is automatically adjusted during treatment. This ensures that the pressure wave 16 can be generated as specified at a desired pressure wave generating pressure P_{D_i}. Furthermore, again according to the sixth step S6, the gas pressure, i.e. the pressure wave generating pressure P_{D_i} and/or the source gas pressure P_{s_i}, is detected in the connection line 22 by means of the pressure sensor 50.

If it is necessary for the treatment with pressure waves, or if the desired pressure wave generating pressure P_{D_i} cannot (any longer) be adjusted by opening/closing in steps S8′, S9′, the power level of the compressor(s) can be “switched” to another stage in a tenth step S10, i.e. the speed at which the compressor(s) is/are operated for the generation of pressure waves 16 is switched to a higher or lower stage. After such a modification according to step S10, an adaption of the pressure wave generating pressure P_{D_i} according to the fifth to tenth steps S5 to S10 can be carried out again in an eleventh step S11.

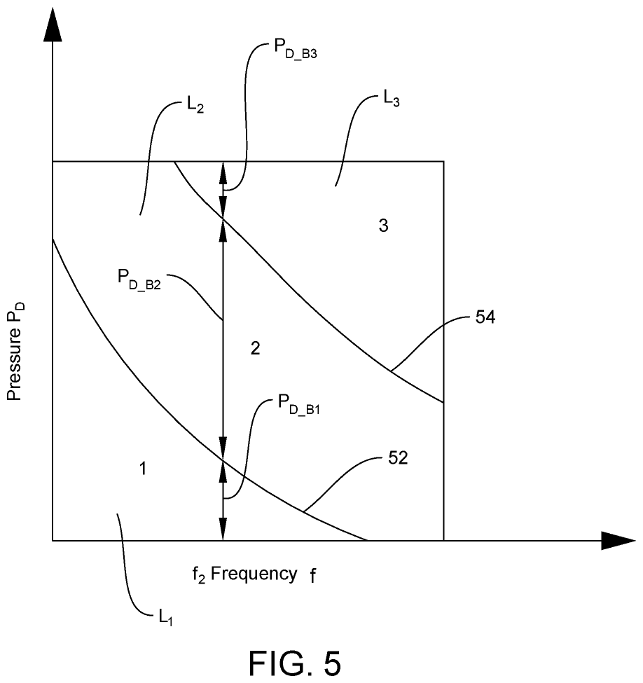

FIG. 5 shows an example of an operating state diagram. For each pair of intended frequency f and intended pressure wave generating pressure P_{D}, a voltage or operating voltage is provided at the compressor. The graphs in FIG. 5 show predetermined first and second switching thresholds 52, 54 for the power levels 1, 2 and 3 with which treatment of the human or animal body with pressure waves 16 is to be carried out. The ranges 1, 2, 3 between the switching thresholds 52, 54 are associated with the discrete speeds, i.e. power levels L_{1,2,3}, adjustable on the at least one compressor 18, 34, 36. If, for example, a second power level L₂ is set, which is assigned to a range 2, it is stored in the control system that a specific second range of pressure wave generating pressures P_{D_B2} can be adjusted at a specific frequency f₂ by means of the pressure regulating device 32. This means that the pressure regulating device 32 is either at least partially opened, or partially closed, or remains unchanged. If, for example, a third power stage L₃ is set, which is assigned to a third range 3, then it is stored in the control system that at the specific frequency f₂ a specific third range of pressure wave generating pressures P_{D_B3} above a second switching threshold 54 can be controlled, i.e. the pressure regulating device 32 can then be at least partially opened or closed in order to adjust the desired pressure wave generating pressure P_{D_i} within this third pressure range P_{D_B3}.

If, for example, a first power stage L₁ is set, which is assigned to a first range 1, then it is stored in the control system that a specific first range of pressure wave generating pressures P_{D_B1} can be controlled at the specific frequency f₂.

In other words: Depending on the predetermined frequency fⱼ, certain ranges of controllable pressure wave generating pressures P_{D_Bi} can be regulated for each power level Lᵢ and depending on the predetermined pressure wave generating pressure P_{D_i}, certain ranges of controllable frequencies f_{Bi} can be regulated for each power level Lᵢ.

In a control system (not shown), corresponding voltage settings U for the compressor(s) 18, 34, 36 are stored for a predetermined switching frequency f and a predetermined pressure wave generating pressure P_{D_i}. According to FIG. 5, it thus results that at a fixed frequency of the pressure wave generating pressure P_{D_i} is changed within the switching thresholds 52, 54 in a power stage (one moves along a vertical line) or at a fixed pressure wave generating pressure P_{D_i} the frequency f can be changed (one moves along a horizontal line). Depending on the frequency f and/or on the pressure wave generating pressure P_{D_i}, the power level L is adjusted by the operation of the compressor(s) 18, 34, 36. Of course, the proposed solution also includes a movement along the graph of FIG. 5, which is composed of a horizontal and vertical movement, i.e. a change in pressure wave generating pressure P_{D} and frequency f.

It is understood that the few ranges 1, 2, 3 and the switching thresholds 52, 54 of FIG. 5 are purely exemplary. It is conceivable that considerably more ranges and switching thresholds are stored in the control, and thus a step module with a plurality of fixed adjustable steps, i.e. discrete stepped power levels L is stored. Further, it is understood that the pressure wave device 10 may have a plurality of compressors 18, wherein each or many compressors 18 may have its/their own compressor motor 20. The use of multiple compressors 18 reduces the noise level.

1. A pressure wave device for the treatment of a human or animal body, comprising:
a pneumatic drive for generating a pressure wave for coupling into the human or animal body,
at least one compressor for generating source gas,
a handpiece into which the source gas can be introduced and by means of which a pressure wave is generated, and
a pressure regulating device for adjusting a pressure wave generating pressure for generating the pressure wave,
wherein the at least one compressor for generating source gas is adjustable in steps to at least two power levels, and
wherein the pressure regulating device regulates the pressure wave generating pressure (P_{D_i}) by adapting the source gas pressure (P_{s_i}) at each power level.

2. The Pressure wave device according to claim 1, wherein each power level is determined by a range of pressure wave generating pressure values and frequencies of the activation of the pressure waves.

3. The pressure wave device according to one of claim 1, wherein the selection of a power level is made on the basis of a table in which the respective ranges of pressure wave generating pressure values and frequencies are stored.

4. The pressure wave device according to claim 1,
wherein the at least one compressor can be switched from a first operating state with a first power level to a second operating state with a second power level in order to set a second pressure wave generating pressure by the pressure regulating device as soon as the pressure wave generating pressure generated in the first operating state is no longer reached at a specific frequency.

5. The pressure wave device according to claim 1,
wherein the pressure regulating device comprises a pressure reducer or a drain valve comprising a valve such as a solenoid valve,
wherein the pressure reducer and/or the drain valve comprises a proportioning valve.

6. The pressure wave device according to claim 1,
wherein the pressure regulating device has at least one pressure sensor for determining a pressure wave generating pressure.

7. The pressure wave device according to claim 1,
wherein at least two compressors are connected to one or are each connected to one motor for controlling the compressors.

8. The pressure wave device according to claim 1,
wherein a cold trap for collecting cooled, condensed source gas is arranged between the at least one compressor and the pressure regulating device.

9. The pressure wave device according to claim 1,
wherein a relief valve or drain valve is provided between the at least one compressor and the pressure regulating device for discharging an overpressure of the source gas.

10. The pressure wave device according to claim 1,
wherein the pressure wave device can be vented by means of a controllable venting valve.

11. A method of treating a human or animal body with pressure waves generated with a pressure wave device according to claim 1, the method comprising the steps of:
starting the at least one compressor to generate source gas,
passing the source gas from the at least one compressor to the handpiece,
wherein the at least one compressor is set to a discretely controllable operating state to which a discrete power level L is assigned,
wherein the pressure wave generating pressure is regulated by adapting a first source gas pressure within the power level L by a pressure regulating device at a certain frequency,
and wherein the pressure wave generating pressure is regulated by increasing or by decreasing the power level L.

12. The method according to claim 11, the method further comprising the steps of:
closing a venting valve, and/or
at least partially opening or at least partially closing a pressure regulating device for passing the source gas from the at least one compressor to a handpiece.

13. The method according to claim 11, wherein after a detection of the actually generated pressure wave generating pressure it is checked whether the detected pressure wave generating pressure corresponds to a desired pressure wave generating pressure and, depending on a difference between detected pressure wave generating pressure and desired pressure wave generating pressure,
a) the state of the pressure regulating device is maintained unchanged, provided that the difference is approximately zero;
b) the pressure regulating device is at least partially opened, provided that the difference is positive, i.e. the detected pressure wave generating pressure is above the desired pressure wave generating pressure, and
c) the pressure regulating device is at least partially closed, provided that the difference is negative, i.e. the detected pressure wave generating pressure is below the desired pressure wave generating pressure.

14. The method according to claim 11, wherein a compressor speed of the at least one compressor is incrementally increased or decreased when P_{s_(i-1)}≤P_{D_i} applies to the maximum source gas pressure or P_{D_i}≤P_{s_(i-1)} applies the pressure wave generating pressure, wherein increasing or decreasing the compressor speed is dependent on the set power level, which is determined by a range of pressure wave generating pressure values and frequencies of the activation of the pressure waves.

15. The method according to claim 11, wherein the setting of the power level and/or the regulation of the pressure wave generating pressure can be changed during a treatment.
